(19) United States
(12) Patent Application Publication
(10) Pub. No.: US 2008/0131490 A1
(43) Pub. Date: Jun. 5, 2008

(54) Stabilized donepezil-containing patch preparation

(76) Inventors: Akinori Hanatani, Osaka (JP); Junichi Sekiya, Osaka (JP); Sachiko Terashi, Osaka (JP); Sumiyo Nishi, Osaka (JP); Satoko Washiro, Osaka (JP); Hitoshi Akemi, Osaka (JP)

Correspondence Address: WENDEROTH, LIND & PONACK, L.L.P., 2033 K STREET N. W., SUITE 800, WASHINGTON, DC, 20006-1021, US

(21) Appl. No.: 11/987,480

(22) Filed: Nov. 30, 2007

Related U.S. Application Data
(60) Provisional application No. 60/861,965, filed on Dec. 1, 2006.

(30) Foreign Application Priority Data
Dec. 1, 2006 (JP) 2006-325104
Jun. 7, 2007 (JP) 2007-152016
Jun. 7, 2007 (JP) 2007-152047

(51) Int. Cl. A61K 9/70; A61K 31/445; A61P 25/28

(52) U.S. Cl. 424/448; 514/319

(57) ABSTRACT

A donepezil-containing patch preparation is provided whereby production of donepezil-related substances in the pressure-sensitive adhesive layer is suppressed. A stabilizer comprising at least one or more species selected from the group consisting of ascorbic acid, metal salts or esters thereof, isoascorbic acid or metal salts thereof, ethylenediamine tetraacetic acid or metal salts thereof, cysteine, acetylcysteine, 2-mercaptobenzimidazole, 3(2)-t-butyl-4-hydroxyanisole, 2,6-di-t-butyl-4-methylphenol, tetrakis[3-(3′,5′-di-t-butyl-4′-hydroxyphenyl)propionic acid]pentaerythritol, 3-mercapto-1,2-propanediol, tocopherol acetate, rutin, quercetin, hydroquinone, metal salts of hydroxymethanesulfinic acid, metal metabisulfite salts, metal sulfite salts and metal thiosulfate salts is blended in a donepezil-containing pressure-sensitive adhesive layer provided on at least one side of a support.

### BACKGROUND

### The present invention relates to a patch preparation containing donepezil.

The basic drug donepezil has acetylcholine esterase inhibitory action, and is used as an anti-Alzheimer's dementia drug. Alzheimer's dementia patients are usually elderly, and elderly patients often have difficulty swallowing oral Dosage forms. In some cases it may also be difficult to administer oral Dosage forms to patients with advanced symptoms of Alzheimer's dementia. In these cases, percutaneous parenteral administration of donepezil is useful.

Patch preparations containing donepezil for purposes of percutaneous parenteral administration of donepezil are known, and are described for example in Japanese Patent Application Laid-open No. H11-315016, WO 2003/032960 pamphlet, WO 2006/082728 pamphlet and the like.

However, a problem encountered by these inventors in our research is that the effective amount of donepezil in the preparation significantly declines over time after a donepezil-containing patch preparation is prepared. Namely, measures need to be taken to prevent production of donepezil-related substances in the patch preparation (pressure-sensitive adhesive layer). However, in a patch preparation for parenteral administration, since the environment of the drug is much different from that in an orally administered preparation, and a preparation in sheet form is especially vulnerable to the effects of the surrounding environment (oxygen), compounding antioxidants (stabilizers) may not stabilize donepezil and large quantities of donepezil-related substances may be produced even if an antioxidant (stabilizer) commonly used in oral Dosage forms is included.

WO 2003/032960 pamphlet and WO 2006/082728 pamphlet describe that antioxidants such as tocopherol and ester derivatives thereof, ascorbic acid, ascorbyl stearate, nordihydroguaiaretic acid, dibutylhydroxytoluene (BHT), butylhydroxyanisole and the like can be used in the pressure-sensitive adhesive layer as necessary, but these do not describe any preparation examples in which antioxidants were actually blended or adequately verify the effectiveness of the antioxidants in the preparations, so no stabilization of donepezil with antioxidants has been discovered.

Japanese Patent Application Laid-open No. 2000-136134 describes that an increase in donepezil-related substances can be inhibited by adding antioxidants such as sodium hydrogensulfite, sodium sulfite, sodium pyrosulfite (sodium metabisulfite), cysteine, citric acid, edetate disodium (disodium ethylenediaminetetraacetate), ascorbic acid and erythorbic acid (isoascorbic acid) and the like to an orally administered composition containing donepezil. However, this document proposes a liquid, syrup or other orally administered preparation, and does not suggest application of antioxidants to patch preparations.

### SUMMARY

Under these circumstances, it is an object of the present invention to reduce the amount of donepezil-related substances produced in a donepezil patch preparation.

As a result of exhaustive research aimed at reducing the produced amounts of specific donepezil-related substances which are found in comparative large amounts in donepezil patch preparations, the inventors in this case discovered stabilizers capable of effectively controlling the produced amounts of these related substances, and perfected the present invention after arriving at the unexpected finding that the total produced amount of related substances could also be reduced thereby.

Namely, the present invention is as follows:

(1) A patch preparation comprising, a support and a pressure-sensitive adhesive layer on at least one side of the support, the pressure-sensitive adhesive layer comprising a pressure-sensitive adhesive, donepezil and a stabilizer,

wherein said stabilizer comprises at least one or more species selected from the group consisting of ascorbic acid, metal salts or esters thereof, isoascorbic acid or metal salts thereof, ethylenediamine tetraacetic acid or metal salts thereof, cysteine, acetylcysteine, 2-mercaptobenzimidazole, 3(2)-t-butyl-4-hydroxyanisole, 2,6-di-t-butyl-4-methylphenol, tetrakis[3-(3′,5′-di-t-butyl-4′-hydroxyphenyl)propionic acid]pentaerythritol, 3-mercapto-1,2-propanediol, tocopherol acetate, rutin, quercetin, hydroquinone, metal salts of hydroxymethanesulfinic acid, metal metabisulfite salts, metal sulfite salts and metal thiosulfate salts.

(2) The patch preparation according to (1) above, wherein said stabilizer comprises at least one or more species selected from the group consisting of ascorbic acid, metal salts or esters thereof, isoascorbic acid or metal salts thereof, ethylenediamine tetraacetic acid or metal salts thereof, cysteine, acetylcysteine, 2-mercaptobenzimidazole, 2,6-di-t-butyl-4-methylphenol, tetrakis[3-(3′,5′-di-t-butyl-4′-hydroxyphenyl)propionic acid]pentaerythritol, 3-mercapto-1,2-propanediol, tocopherol acetate, rutin, quercetin, metal salts of hydroxymethanesulfinic acid, metal metabisulfite salts, metal sulfite salts and metal thiosulfate salts.

(3) The patch preparation according to (1) above, wherein said stabilizer comprises at least one or more species selected from the group consisting of ascorbic acid, metal salts or esters thereof, isoascorbic acid or metal salts thereof, ethylenediamine tetraacetic acid or metal salts thereof, 2-mercaptobenzimidazole, 2,6-di-t-butyl-4-methylphenol, tetrakis[3-(3′,5′-di-t-butyl-4′-hydroxyphenyl)propionic acid]pentaerythritol, tocopherol acetate, rutin, metal sulfite salts and metal thiosulfate salts.

(4) The patch preparation according to (1) above, wherein said stabilizer comprises at least one or more species selected from the group consisting of ascorbic acid, metal salts or esters thereof, isoascorbic acid or metal salts thereof, ethylenediamine tetraacetic acid or metal salts thereof, 2-mercaptobenzimidazole, 3(2)-t-butyl-4-hydroxyanisole, 2,6-di-t-butyl-4-methylphenol, tetrakis[3-(3′,5′-di-t-butyl-4′-hydroxyphenyl)propionic acid]pentaerythritol, tocopherol acetate, rutin, quercetin, hydroquinone and metal thiosulfate salts.

(5) The patch preparation according to (1) above, wherein said stabilizer comprises at least one or more species selected from the group consisting of ascorbic acid, metal salts or esters thereof, isoascorbic acid or metal salts, ethylenediamine tetraacetic acid or metal salts thereof, 2-mercaptobenzimidazole, 2,6-di-t-butyl-4-methylphenol, tetrakis[3-(3′,5′-di-t-butyl-4′-hydroxyphenyl)propionic acid]pentaerythritol, tocopherol acetate, rutin and metal thiosulfate salts.

(6) The patch preparation according to (1) above, wherein said stabilizer comprises at least one or more species selected from the group consisting of ascorbic acid, metal salts or esters thereof, isoascorbic acid or metal salts thereof, 2-mercaptobenzimidazole, 2,6-di-t-butyl-4-methylphenol, metal salt of hydroxymethanesulfinic acid, rutin and metal metabisulfite salts.

(7) The patch preparation according to (1) above, wherein said stabilizer comprises:

ascorbic acid, metal salts or esters thereof and metal metabisulfite salts, or

ascorbic acid, metal salts or esters thereof and 2-mercaptobenzimidazole.

(8) The patch preparation according to (1) above, wherein said stabilizer comprises at least one or more species selected from the group consisting of ascorbic acid, metal salts or esters thereof, 2-mercaptobenzimidazole, metal metabisulfite salts and metal sulfite salts.

(9) A patch preparation comprising, a support and a pressure-sensitive adhesive layer on at least one side of the support, the pressure-sensitive adhesive layer comprising donepezil, wherein said pressure-sensitive adhesive layer is obtainable by forming a film of a mixture comprising a pressure-sensitive adhesive, donepezil and a stabilizer,

and wherein said stabilizer comprises at least one or more species selected from the group consisting of ascorbic acid, metal salts or esters thereof, isoascorbic acid or metal salts thereof, ethylenediamine tetraacetic acid or metal salts thereof, cysteine, acetylcysteine, 2-mercaptobenzimidazole, 3(2)-t-butyl-4-hydroxyanisole, 2,6-di-t-butyl-4-methylphenol, tetrakis[3-(3′,5′-di-t-butyl-4′-hydroxyphenyl)propionic acid]pentaerythritol, 3-mercapto-1,2-propanediol, tocopherol acetate, rutin, quercetin, hydroquinone, metal salts of hydroxymethanesulfinic acid, metal metabisulfite salts, metal sulfite salts and metal thiosulfate salts.

(10) The patch preparation according to (9) above, wherein said stabilizer comprises at least one or more species selected from the group consisting of ascorbic acid, metal salts or esters thereof, isoascorbic acid or metal salts thereof, ethylenediamine tetraacetic acid or metal salts thereof, cysteine, acetylcysteine, 2-mercaptobenzimidazole, 2,6-di-t-butyl-4-methylphenol, tetrakis[3-(3′,5′-di-t-butyl-4′-hydroxyphenyl)propionic acid]pentaerythritol, 3-mercapto-1,2-propanediol, tocopherol acetate, rutin, quercetin, metal salts of hydroxymethanesulfinic acid, metal metabisulfite salts, metal sulfite salts and metal thiosulfate salts.

(11) The patch preparation according to (9) above, wherein said stabilizer comprises at least one or more species selected from the group consisting of ascorbic acid, metal salts or esters thereof, isoascorbic acid or metal salts thereof, ethylenediamine tetraacetic acid or metal salts thereof, 2-mercaptobenzimidazole, 2,6-di-t-butyl-4-methylphenol, tetrakis[3-(3′,5′-di-t-butyl-4′-hydroxyphenyl)propionic acid]pentaerythritol, tocopherol acetate, rutin, metal sulfite salts and metal thiosulfate salts.

(12) The patch preparation according to (9) above, wherein said stabilizer comprises at least one or more species selected from the group consisting of ascorbic acid, metal salts or esters thereof, isoascorbic acid or metal salts thereof, ethylenediamine tetraacetic acid or metal salts thereof, 2-mercaptobenzimidazole, 3(2)-t-butyl-4-hydroxyanisole, 2,6-di-t-butyl-4-methylphenol, tetrakis[3-(3′,5′-di-t-butyl-4′-hydroxyphenyl)propionic acid]pentaerythritol, tocopherol acetate, rutin, quercetin, hydroquinone and metal thiosulfate salts.

(13) The patch preparation according to (9) above, wherein said stabilizer comprises at least one or more species selected from the group consisting of ascorbic acid, metal salts or esters thereof, isoascorbic acid or metal salts, ethylenediamine tetraacetic acid or metal salts thereof, 2-mercaptobenzimidazole, 2,6-di-t-butyl-4-methylphenol, tetrakis[3-(3′,5′-di-t-butyl-4′-hydroxyphenyl)propionic acid]pentaerythritol, tocopherol acetate, rutin and metal thiosulfate salts.

(14) The patch preparation according to (9) above, wherein said stabilizer comprises at least one or more species selected from the group consisting of ascorbic acid, metal salts or esters thereof, isoascorbic acid or metal salts thereof, 2-mercaptobenzimidazole, 2,6-di-t-butyl-4-methylphenol, metal salts of hydroxymethanesulfinic acid, rutin and metal metabisulfite salts.

(15) The patch preparation according to (9) above, wherein said stabilizer comprises:

ascorbic acid, metal salts or esters thereof and metal metabisulfite salts, or

ascorbic acid, metal salts or esters thereof and 2-mercaptobenzimidazole.

(16) The patch preparation according to (9) above, wherein said stabilizer comprises at least one or more species selected from the group consisting of ascorbic acid, metal salts or esters thereof, 2-mercaptobenzimidazole, metal metabisulfite salts and metal sulfite salts.

(17) A method for stabilizing donepezil in a patch preparation, comprising the step of including at least one or more species selected from the group consisting of ascorbic acid, metal salts or esters thereof, isoascorbic acid or metal salts thereof, ethylenediamine tetraacetic acid or metal salts thereof, cysteine, acetylcysteine, 2-mercaptobenzimidazole, 3(2)-t-butyl-4-hydroxyanisole, 2,6-di-t-butyl-4-methylphenol, tetrakis[3-(3′,5′-di-t-butyl-4′-hydroxyphenyl)propionic acid]pentaerythritol, 3-mercapto-1,2-propanediol, tocopherol acetate, rutin, quercetin, hydroquinone, metal salts of hydroxymethanesulfinic acid, metal metabisulfite salts, metal sulfite salts and metal thiosulfate salts together with donepezil in the presence of a pressure-sensitive adhesive.

(A) A patch preparation comprising, a support and a pressure-sensitive adhesive layer on at least one side of the support, the pressure-sensitive adhesive layer comprising a pressure-sensitive adhesive, donepezil and a stabilizer,

wherein the stabilizer comprises at least one or more compound combinations selected from the following group of compound combinations (a) through (j):

(a) isoascorbic acid or metal salts thereof and 2-mercaptobenzimidazole;

(b) isoascorbic acid or metal salts thereof and 2,6-di-t-butyl-4-methylphenol;

(c) isoascorbic acid or metal salts thereof and metal salts of hydroxymethanesulfinic acid;

(d) isoascorbic acid or metal salts thereof and rutin;

(e) 2-mercaptobenzimidazole and 2,6-di-t-butyl-4-methylphenol;

(f) 2-mercaptobenzimidazole and metal salts of hydroxymethanesulfinic acid;

(g) 2-mercaptobenzimidazole and rutin;

(h) 2,6-di-t-butyl-4-methylphenol and metal salts of hydroxymethanesulfinic acid;

(i) 2,6-di-t-butyl-4-methylphenol and rutin;

(j) metal salts of hydroxymethanesulfinic acid and rutin.

(B) The patch preparation according to (A) above, wherein the stabilizer comprises at least one or more compound combinations selected from the following group of compound combinations (a) through (e) and (g) through (j):

(a) isoascorbic acid or metal salts thereof and 2-mercaptobenzimidazole;

(b) isoascorbic acid or metal salts thereof and 2,6-di-t-butyl-4-methylphenol;

(c) isoascorbic acid or metal salts thereof and metal salts of hydroxymethanesulfinic acid;

(d) isoascorbic acid or metal salts thereof and rutin;

(e) 2-mercaptobenzimidazole and 2,6-di-t-butyl-4-methylphenol;

(g) 2-mercaptobenzimidazole and rutin;

(h) 2,6-di-t-butyl-4-methylphenol and metal salt of hydroxymethanesulfinic acid;

(i) 2,6-di-t-butyl-4-methylphenol and rutin;

(j) metal salt of hydroxymethanesulfinic acid and rutin.

(C) The patch preparation according to (A) above, wherein the stabilizer comprises at least one or more compound combinations selected from the following group of compound combinations (a), (b), (d), (e) and (g) through (i):

(a) isoascorbic acid or metal salts thereof and 2-mercaptobenzimidazole;

(b) isoascorbic acid or metal salts thereof and 2,6-di-t-butyl-4-methylphenol;

(d) isoascorbic acid or metal salts thereof and rutin;

(e) 2-mercaptobenzimidazole and 2,6-di-t-butyl-4-methylphenol;

(g) 2-mercaptobenzimidazole and rutin;

(h) 2,6-di-t-butyl-4-methylphenol and metal salts of hydroxymethanesulfinic acid;

(i) 2,6-di-t-butyl-4-methylphenol and rutin.

(D) The patch preparation according to (A) above, wherein the stabilizer comprises at least one or more compound combinations selected from the following group of compound combinations (a), (c), (e), (g), (h) and (j)

(a) isoascorbic acid or metal salts thereof and 2-mercaptobenzimidazole;

(c) isoascorbic acid or metal salts thereof and metal salt of hydroxymethanesulfinic acid;

(e) 2-mercaptobenzimidazole and 2,6-di-t-butyl-4-methylphenol;

(g) 2-mercaptobenzimidazole and rutin;

(h) 2,6-di-t-butyl-4-methylphenol and metal salts of hydroxymethanesulfinic acid;

(j) metal salts of hydroxymethanesulfinic acid and rutin.

(e) The patch preparation according to (A) above, wherein the stabilizer comprises at least one or more compound combinations selected from the following group of compound combinations (c), (e), (h) and (j) (c) isoascorbic acid or metal salts thereof and metal salt of hydroxymethanesulfinic acid

(e) 2-mercaptobenzimidazole and 2,6-di-t-butyl-4-methylphenol;

(h) 2,6-di-t-butyl-4-methylphenol and metal salts of hydroxymethanesulfinic acid;

(j) metal salts of hydroxymethanesulfinic acid and rutin.

(F) A patch preparation comprising, a support and a pressure-sensitive adhesive layer on at least one side of the support, the pressure-sensitive adhesive layer comprising donepezil, wherein said pressure-sensitive adhesive layer is obtainable by forming a film of a mixture comprising a pressure-sensitive adhesive, donepezil and a stabilizer,

and wherein said stabilizer comprises at least one or more compound combinations selected from the following group of compound combinations (a) through (j):

(a) isoascorbic acid or metal salts thereof and 2-mercaptobenzimidazole;

(b) isoascorbic acid or metal salts thereof and 2,6-di-t-butyl-4-methylphenol;

(c) isoascorbic acid or metal salts thereof and metal salts of hydroxymethanesulfinic acid;

(d) isoascorbic acid or metal salts thereof and rutin;

(e) 2-mercaptobenzimidazole and 2,6-di-t-butyl-4-methylphenol;

(f) 2-mercaptobenzimidazole and metal salts of hydroxymethanesulfinic acid;

(g) 2-mercaptobenzimidazole and rutin,

(h) 2,6-di-t-butyl-4-methylphenol and metal salts of hydroxymethanesulfinic acid;

(i) 2,6-di-t-butyl-4-methylphenol and rutin;

metal salts of hydroxymethanesulfinic acid and rutin.

(G) The patch preparation according to (F) above, wherein the stabilizer comprises at least one or more compound combinations selected from the following group of compound combinations (a) through (e) and (g) through (j):

(a) isoascorbic acid or metal salts thereof and 2-mercaptobenzimidazole;

(b) isoascorbic acid or metal salts thereof and 2,6-di-t-butyl-4-methylphenol;

(c) isoascorbic acid or metal salts thereof and metal salts of hydroxymethanesulfinic acid;

(d) isoascorbic acid or metal salts thereof and rutin;

(e) 2-mercaptobenzimidazole and 2,6-di-t-butyl-4-methylphenol;

(g) 2-mercaptobenzimidazole and rutin;

(h) 2,6-di-t-butyl-4-methylphenol and metal salts of hydroxymethanesulfinic acid;

(i) 2,6-di-t-butyl-4-methylphenol and rutin;

(j) metal salts of hydroxymethanesulfinic acid and rutin.

(H) The patch preparation according to (F) above, wherein the stabilizer comprises at least one or more compound combinations selected from the following group of compound combinations (a), (b), (d), (e) and (g) through (i):

(a) isoascorbic acid or metal salts thereof and 2-mercaptobenzimidazole;

(b) isoascorbic acid or metal salts thereof and 2,6-di-t-butyl-4-methylphenol;

(d) isoascorbic acid or metal salts thereof and rutin;

(e) 2-mercaptobenzimidazole and 2,6-di-t-butyl-4-methylphenol;

(g) 2-mercaptobenzimidazole and rutin;

(h) 2,6-di-t-butyl-4-methylphenol and metal salts of hydroxymethanesulfinic acid;

(i) 2,6-di-t-butyl-4-methylphenol and rutin.

(l) The patch preparation according to (F) above, wherein the stabilizer comprises at least one or more compound combinations selected from the following group of compound combinations (a), (c), (e), (g), (h) and (j)

(a) isoascorbic acid or metal salts thereof and 2-mercaptobenzimidazole;

(c) isoascorbic acid or metal salts thereof and metal salt of hydroxymethanesulfinic acid

(e) 2-mercaptobenzimidazole and 2,6-di-t-butyl-4-methylphenol;

(g) 2-mercaptobenzimidazole and rutin;

(h) 2,6-di-t-butyl-4-methylphenol and metal salts of hydroxymethanesulfinic acid;

(j) metal salts of hydroxymethanesulfinic acid and rutin.

(J) The patch preparation according to (F) above, wherein the stabilizer comprises at least one or more compound combinations selected from the following group of compound combinations (c), (e), (h) and (j)

(c) isoascorbic acid or metal salts thereof and metal salt of hydroxymethanesulfinic acid

(e) 2-mercaptobenzimidazole and 2,6-di-t-butyl-4-methylphenol;

(h) 2,6-di-t-butyl-4-methylphenol and metal salts of hydroxymethanesulfinic acid;

j) metal salts of hydroxymethanesulfinic acid and rutin.

(K) A patch preparation comprising, a support and a pressure-sensitive adhesive layer on at least one side of the support, the pressure-sensitive adhesive layer comprising a pressure-sensitive adhesive, donepezil and a stabilizer,

wherein the stabilizer is a combination of any 2 selected from the group consisting of 2-mercaptobenzimidazole, the metal metabisulfite salts and the metal sulfite salts.

(L) A patch preparation comprising, a support and a pressure-sensitive adhesive layer on at least one side of the support, the pressure-sensitive adhesive layer comprising donepezil, wherein said pressure-sensitive adhesive layer is obtainable by forming a film of a mixture comprising a pressure-sensitive adhesive, donepezil and a stabilizer,

and wherein the stabilizer is a combination of any 2 selected from the group consisting of 2-mercaptobenzimidazole, the metal metabisulfite salts and the metal sulfite salts.

According to the present invention it is possible to reliably reduce the produced amounts of specific donepezil-related substances in a preparation to thereby achieve a highly safe and stable donepezil-containing patch preparation.

With a preferred embodiment it is possible to reduce not only the produced amounts of specific related substances but also the total produced amount of related substances in the preparation to thereby achieve an extremely safe and stable donepezil-containing patch preparation.

Moreover, by using a combination of two specific stabilizers it is possible to reliably reduce the produced amounts of specific donepezil-related substances in a preparation to thereby achieve a highly safe and stable donepezil-containing patch preparation.

In a preferred embodiment, the produced amount of related substances is further reduced below the arithmetic mean value of the produced amounts obtained with each stabilizer independently by using two or more stabilizers in combination (namely, there is a synergistic effect). Consequently, a highly safe and stable donepezil-containing patch preparation can be efficiently obtained without the need for large quantities of stabilizers.

### DETAILED DESCRIPTION

Preferred embodiments of the present invention are explained below.

The patch preparation of the present invention comprises a support and a pressure-sensitive adhesive layer formed on at least one side of that support, and the pressure-sensitive adhesive layer comprises at least donepezil, a pressure-sensitive adhesive and a stabilizer.

The term “donepezil” here encompasses not only (±)-2-[(1-benzylpiperidin-4-yl)methyl]-5,6-dimethoxyindan-1-one (the free form), but also pharmacologically acceptable salts and esters thereof.

The term “stabilizer” signifies a compound that may have the action of suppressing production of donepezil-related substances (or reducing the produced amounts of such related substances) in a pressure-sensitive adhesive layer containing donepezil and in the mixture of materials used to form such a pressure-sensitive adhesive layer.

The donepezil in the present invention may be either donepezil (the free form) or any of pharmaceutically acceptable salts or esters thereof, but the pressure-sensitive adhesive layer should preferably contain donepezil (the free form) from the standpoint of percutaneous absorbability.

The patch preparation of the present invention can be used as an anti-Alzheimer's dementia drug. Other possible applications include for cerebrovascular dementia, for prevention of migraine and the like.

In the patch preparation of the present invention, the proportion of donepezil in the pressure-sensitive adhesive layer is preferably 1 to 30 wt % or more preferably 3 to 20 wt % based on the total weight of the pressure-sensitive adhesive layer. If the proportion is less than 1 wt % enough may not be released to be clinically effective, while more than 30 wt % is uneconomical without providing any further clinical benefits.

The stabilizer used in the present invention is a specific stabilizer which is chosen from the group consisting of ascorbic acid, metal salts or esters thereof (preferably sodium salt or palmitic acid ester), isoascorbic acid or metal salts thereof (preferably sodium salt), ethylenediamine tetraacetic acid or metal salts thereof (preferably calcium disodium salt or tetrasodium salt), cysteine, acetylcysteine, 2-mercaptobenzimidazole, 3(2)-t-butyl-4-hydroxyanisole, 2,6-di-t-butyl-4-methylphenol, tetrakis[3-(3′,5′-di-t-butyl-4′-hydroxyphenyl)propionic acid]pentaerythritol, 3-mercapto-1,2-propanediol, tocopherol acetate, rutin, quercetin, hydroquinone and the metal salts of hydroxymethanesulfinic acid (preferably sodium salts), metal metabisulfite salts (preferably sodium salts), metal sulfite salts (preferably sodium salts) and metal thiosulfate salts (preferably sodium salts), and one of these may be used or two or more may be used in combination.

Examples of the aforementioned metals salts include sodium salts, potassium salts, calcium salts, magnesium salts and the like. Examples of esters include palmitic acid esters, stearic acid esters, myristic acid esters and the like.

The stabilizer must be contained in the pressure-sensitive adhesive layer of the patch preparation. The weight proportion of the stabilizer is not particularly limited as long as it does not adversely affect the properties of the pressure-sensitive adhesive layer. Regarding the maximum percentage of stabilizer based on the total weight of the pressure-sensitive adhesive layer (namely, the total solids weight of the mixture used to form the pressure-sensitive adhesive layer), if the percentage is above 5 wt % of the total the adhesiveness and other properties of the pressure-sensitive adhesive layer may be adversely affected, while below 0.0005 wt % a sufficient stabilizing effect may not be obtained. Consequently, preferred examples of the maximum percentage are 5 wt %, 3 wt %, 2 wt %, 1 wt %, 0.7 wt %, 0.5 wt % and 0.3 wt %, while preferred examples of the minimum percentage are 0.0005 wt %, 0.001 wt %, 0.01 wt %, 0.02 wt %, 0.03 wt %, 0.05 wt %, 0.1 wt % and 0.2 wt %.

More specifically, the percentage is preferably 0.0005 to 5 wt % or more preferably 0.001 to 3 wt % or still more preferably 0.01 to 1 wt % or still more preferably 0.01 to 0.91 wt % or still more preferably 0.01 to 0.7 wt % or still more preferably 0.02 to 0.7 wt % or still more preferably 0.02 to 0.5 wt % or ideally 0.03 to 0.3 wt % based on the total weight of the pressure-sensitive adhesive layer (the total solids weight of the mixture used to form the pressure-sensitive adhesive layer).

In the present invention, “donepezil-related substances” are substances that are found in comparatively large amounts in pressure-sensitive adhesive layers containing donepezil and that are derived from donepezil (substances produced in association with donepezil; substances not found in pressure-sensitive adhesive layers not containing donepezil), and specifically are the related substance detected with a retention time of 12.8 minutes (hereinafter called “related substance 1”) and the related substance detected with a retention time of 3.9 minutes (hereinafter called “related substance 2”) when the patch preparation of the present invention was analyzed under the analysis conditions described in the examples below. It is a principal object of the present invention to at least suppress the production of this specific related substance 1 and/or related substance 2, and this object is achieved by including at least one or two or more specific stabilizers in the pressure-sensitive adhesive layer.

Of the aforementioned stabilizers, those most desirable for efficiently reducing the produced amount of donepezil-related substance 1 are ascorbic acid, metal salts or esters thereof, isoascorbic acid or metal salts thereof, ethylenediamine tetraacetic acid or metal salts thereof, cysteine, acetylcysteine, 2-mercaptobenzimidazole, 2,6-di-t-butyl-4-methylphenol, tetrakis[3-(3′,5′-di-t-butyl-4′-hydroxyphenyl)propionic acid]pentaerythritol, 3-mercapto-1,2-propanediol, tocopherol acetate, rutin, quercetin and the metal salts of hydroxymethanesulfinic acid, metal metabisulfite salts, metal sulfite salts and metal thiosulfate salts, and one of these may be used or two or more may be used in combination.

Even if production of related substance 1 is suppressed, more of other related substances may be produced and the total produced amount of related substances may rise as a result. However, the inventors in this case have discovered that not only the produced amount of related substance 1 but also the total produced amount of related substances can be reduced by selecting certain kinds of stabilizers.

Such stabilizers are preferably ascorbic acid, metal salts or esters thereof, isoascorbic acid or metal salts thereof, ethylenediamine tetraacetic acid or metal salts thereof, 2-mercaptobenzimidazole, 2,6-di-t-butyl-4-methylphenol, tetrakis[3-(3′,5′-di-t-butyl-4′-hydroxyphenyl)propionic acid]pentaerythritol, tocopherol acetate, rutin and the metal sulfite salts and metal thiosulfate salts, and one of these may be used or two or more may be used in combination.

Of the aforementioned stabilizers, those most desirable for efficiently reducing the produced amount of donepezil-related substance 2 are ascorbic acid, metal salts or esters thereof, isoascorbic acid or metal salts thereof, ethylenediamine tetraacetic acid or metal salts thereof, 2-mercaptobenzimidazole, 3(2)-t-butyl-4-hydroxyanisole, 2,6-di-t-butyl-4-methylphenol, tetrakis[3-(3′,5′-di-t-butyl-4′-hydroxyphenyl)propionic acid]pentaerythritol, tocopherol acetate, rutin, quercetin, hydroquinone and the metal thiosulfate salts, and one of these may be used or two or more may be used in combination.

Even if production of related substance 2 is suppressed, more of other related substances may be produced and the total produced amount of related substances may rise as a result. However, the inventors in this case have discovered that not only the produced amount of related substance 2 but also the total produced amount of related substances can be reduced by selecting certain kinds of stabilizers.

Such stabilizers are preferably ascorbic acid, metal salts or esters thereof, isoascorbic acid, ethylenediamine tetraacetic acid or metal salts thereof, 2-mercaptobenzimidazole, 2,6-di-t-butyl-4-methylphenol, tetrakis[3-(3′,5′-di-t-butyl-4′-hydroxyphenyl)propionic acid]pentaerythritol, tocopherol acetate, rutin and the metal thiosulfate salts, and one of these may be used or two or more may be used in combination.

Thus, one stabilizer may be used in the present invention or two or more may be used in combination, but in particular a combination of ascorbic acid, metal salts or esters thereof (hereinafter called generally “ascorbic acid derivatives”) with at least one or more stabilizers selected from the following stabilizer group (A) is advantageous because it allows less ascorbic acid, metal salts or esters thereof to be used.

Stabilizer group (A): isoascorbic or metal salts thereof, 2-mercaptobenzimidazole, 2,6-di-t-butyl-4-methylphenol, metal salts of hydroxymethanesulfinic acid, rutin, metal metabisulfite salts

From the standpoint of synergistically suppressing production of related substance 1, the stabilizer selected from stabilizer group (A) is preferably selected from the group consisting of 2-mercaptobenzimidazole, 2,6-di-t-butyl-4-methylphenol, the metal salts of hydroxymethanesulfinic acid and the metal metabisulfite salts.

In an especially preferred embodiment, the combination of ascorbic acid derivatives with another stabilizer is a combination of ascorbic acid derivatives with 2-mercaptobenzimidazole, and with this embodiment it is possible to synergistically suppress not only related substance 1 but also the produced amount of total related substances. Other examples are a combination of ascorbic acid derivatives with metal salts of hydroxymethanesulfinic acid and a combination of ascorbic acid derivatives with metal metabisulfite salts, and with either of these embodiments it is possibly to synergistically suppress the produced amounts of related substance 1, related substance 2 and total related substances.

In the embodiment described above of a combination of ascorbic acid derivatives and another stabilizer, the weight ratio of the two (ascorbic acid derivatives:other stabilizer) is preferably 100:1 to 1:100 or more preferably 10:1 to 1:100 or still more preferably 1:1 to 1:100. If the proportion of ascorbic acid derivatives is raised beyond 100:1 the adhesiveness of the pressure-sensitive adhesive layer may be adversely affected, while if the proportion of the other stabilizer is raised beyond 1:100 it may not be possible to obtain a good stabilizing effect.

In addition to the aforementioned embodiment combining ascorbic acid derivatives with another stabilizer, the following compound combinations are other possible combinations of stabilizers in the present invention:

(a) isoascorbic acid or metal salts thereof (preferably sodium salt) and 2-mercaptobenzimidazole;

(b) isoascorbic acid or metal salts thereof (preferably sodium salt) and 2,6-di-t-butyl-4-methylphenol;

(c) isoascorbic acid or metal salts thereof (preferably sodium salt) and metal salts of hydroxymethanesulfinic acid (preferably sodium salt);

(d) isoascorbic acid or metal salts thereof (preferably sodium salt) and rutin;

(e) 2-mercaptobenzimidazole and 2,6-di-t-butyl-4-methylphenol;

(f) 2-mercaptobenzimidazole and metal salts of hydroxymethanesulfinic acid;

(g) 2-mercaptobenzimidazole and rutin;

(h) 2,6-di-t-butyl-4-methylphenol and metal salts of hydroxymethanesulfinic acid;

(i) 2,6-di-t-butyl-4-methylphenol and rutin;

(j) metal salts of hydroxymethanesulfinic acid and rutin, and one such compound combination may be used or two or more may be selected and used from this group of compound combinations.

In the other combinations described above, the metal salts may be sodium salts, potassium salts, calcium salts, magnesium salts or the like.

By using the stabilizers (combinations of 2 compounds) given as examples in the other combinations above, it is possible to suppress production of at least related substance 1. However, even if production of related substance 1 is suppressed, more of other related substances may be produced and the total produced amount of related substances (total produced amount of related substance 1, related substance 2 and other related substances) may rise as a result. However, the inventors in this case have discovered that when certain combinations of compounds are selected, it is possible not only to efficiently reduce production of related substance 1 but also to reduce the total production of related substances. The compound combinations of this preferred embodiment are the combinations (a), (b), (c), (d), (e), (g), (h), (i) and (j) out of the 2-compound combinations (a) through (j) above. One such compound combination or two or more may be selected and used.

Compound combinations of a preferred embodiment that efficiently reduces the produced amount of related substance 2 as well as related substance 1 are the combinations (a), (b), (d), (e), (g), (h) and (i) out of the 2-compound combinations (a) through (j) above. One such compound combination or two or more may be selected and used.

Even if production of related substance 2 is suppressed in addition to that of related substance 1, however, more of other related substances (related substance other than related substance 1 and related substance 2) may be produced, and total production of related substances may rise as a result. However, the inventors in this case have discovered that when certain combinations of compounds are selected, it is possible not only to efficiently reduce production of related substance 1 and related substance 2, but also to reduce the total production of related substances. The compound combinations of this preferred embodiment are the combinations (a), (b), (d), (e), (g), (h) and (i) out of the 2-compound combinations (a) through (j) above. One such compound combination or two or more may be selected and used.

Of the 2-compound combinations (a) through (j) above, some combinations are particularly desirable because they have a synergistic reduction effect on the produced amount of related substance 1 which is greater than the arithmetic mean of the reductions in the produced amount of related substance 1 produced by the compounds individually, which means that the amount of stabilizer in the preparation can be reduced. Compound combinations of this particularly preferred embodiment are (a), (c), (e), (g), (h) and (j), and one such combination may be used or two or more may be selected.

Of the 2-compound combinations, there are also combinations which are especially desirable because they not only have a synergistic reduction effect on the produced amount of related substance 1 which is greater than the arithmetic mean of the reductions in the produced amount of related substance 1 produced by the compounds individually, but also have a synergistic reduction effect on the produced amount of total related substances which is greater than the arithmetic means of the reductions in the produced amount of total related substances produced by the compounds individually, which means that the amount of stabilizer in the preparation can be greatly reduced. Compound combinations of this especially preferred embodiment are (c), (e), (h) and (j), and one such combination may be used or two or more may be selected.

Moreover, of the 2-compound combinations there are also combinations which are especially desirable because they have a synergistic reduction effect on the produced amount of related substance 2 which is greater than the arithmetic mean of the reductions in the produced amount of related substance 2 produced by the compounds individually, which means that the amount of stabilizer in the preparation can be reduced. Compound combinations of this particularly preferred embodiment are (c) and (h), and either of these or both may be used.

In addition, of the 2-compound combinations there are also combinations that are even more desirable because they not only have a synergistic reduction effect on the produced amount of related substance 2 which is greater than the arithmetic mean of the reductions in the produced amount of related substance 2 produced by the compounds individually, but also have a synergistic reduction effect on the produced amount of total related substances which is greater than the arithmetic means of the reductions in the produced amount of total related substances produced by the compounds individually, which means that the amount of stabilizer in the preparation can be greatly reduced. Compound combinations of this especially preferred embodiment are (c) and (h), and either of these or both may be used.

In the present invention the blended proportions (weight ratio) of the compounds in the 2-compound combinations ((a) through j)) used for the stabilizer are not particularly limited, but from the standpoint of obtaining a good donepezil-stabilizing effect the ratio should be in the range of 10000:1 to 1:10000 or preferably 1000:1 to 1:1000 or more preferably 100:1 to 1:100 or still more preferably 10:1 to 1:10.

A combination of two selected from the group consisting of 2-mercaptobenzimidazole, the metal metabisulfite salts and the metal sulfite salts can also be used favorably as the stabilizer in the present invention.

In the patch preparation of the present invention, the pressure-sensitive adhesive contained in the pressure-sensitive adhesive layer is not particularly limited, and examples include acrylic pressure-sensitive adhesives; silicone rubber, polyisoprene rubber, polyisobutylene rubber, styrene-butadiene rubber, styrene-isoprene-styrene block copolymer rubber, styrene-butadiene-styrene block copolymer rubber and other rubber pressure-sensitive adhesives; silicone pressure-sensitive adhesives; and polyvinyl alcohol, polyvinyl alkyl ether, polyvinyl acetate and other vinyl polymer pressure-sensitive adhesives and the like.

Rubber pressure-sensitive adhesives usually do not contain highly reactive functional groups, and so the donepezil contained therein is comparatively stable and comparatively few related substances are produced. Examples of such rubber pressure-sensitive adhesives include polyisobutylene, styrene-diene-styrene block copolymers (such as styrene-butadiene-styrene block copolymer (SBS) and styrene-isoprene-styrene block copolymer (SIS), etc.) and the like, and one of these or a mixture of two or more may be used.

Depending on the types and proportions of the copolymerized monomers, acrylic pressure-sensitive adhesives allow control over the degree of drug solubility and are also comparatively adaptable in terms of their adhesive properties and the like, but their polymer chains may contain functional groups that are reactive with donepezil, and residual monomers and polymerization initiators in the pressure-sensitive adhesive may also react with the donepezil, potentially reducing the effective dose of donepezil. Consequently, the present invention is particularly useful in the case of a patch preparation using an acrylic pressure-sensitive adhesive.

An acrylic pressure-sensitive adhesive in the present invention may be an acrylic pressure-sensitive adhesive containing a (meth)acrylic acid alkyl ester, and is preferably an acrylic pressure-sensitive adhesive having a (meth)acrylic acid alkyl ester as a principal component (principal constituent unit). As used herein, the term (meth)acrylic shall mean acrylic or methacrylic. A copolymer of a (meth)acrylic acid alkyl ester (first monomer component) as the principal component with a vinyl monomer having functional groups capable of contributing to a crosslinking reaction (second monomer component) or a copolymer of these copolymerized with yet another monomer (third monomer component) is particularly desirable from the standpoint of ease of crosslinking, pressure-sensitive adhesiveness with human skin, ability to manipulate drug dissolution and the like.

Examples of this (meth)acrylic acid alkyl ester (first monomer component) include (meth)acrylic acid alkyl esters and the like wherein the alkyl group is a straight-chain, branched or cyclic alkyl group having 1 to 18 carbon atoms (such as methyl, ethyl, propyl, butyl, pentyl, hexyl, cyclohexyl, heptyl, octyl, 2-ethylhexyl, nonyl, decyl, undecyl, dodecyl, tridecyl, etc.), and a (meth)acrylic acid alkyl ester wherein the alkyl group is a straight-chain, branched or cyclic alkyl group having 4 to 18 carbon atoms (such as butyl, pentyl, hexyl, cyclohexyl, heptyl, octyl, 2-ethylhexyl, nonyl, decyl, undecyl, dodecyl, tridecyl or the like) is preferred. Since a monomer component that lowers the glass transition temperature of the polymer is particularly desirable for contributing room-temperature adhesiveness, a (meth)acrylic acid alkyl ester wherein the alkyl group is a straight-chain, branched or cyclic alkyl group having 4 to 8 carbon atoms (such as butyl, pentyl, hexyl, cyclohexyl, heptyl, octyl, 2-ethylhexyl or the like or preferably butyl, 2-ethylhexyl or cyclohexyl or most preferably 2-ethylhexyl) is more preferred. Specifically, butyl acrylate, 2-ethylhexyl acrylate, 2-ethylhexyl methacrylate, cyclohexyl acrylate, cyclohexyl methacrylate or the like is desirable, and of these, 2-ethylhexyl acrylate is particularly desirable. One such (meth)acrylic acid alkyl ester (first monomer component) may be used, or two or more may be used in combination.

In the vinyl monomer (second monomer component) having functional groups capable of contributing to the crosslinking reaction, the functional groups capable of contributing to the crosslinking reaction may be hydroxyl groups, carboxyl groups, vinyl groups or the like, and hydroxyl groups and carboxyl groups are preferred. Specific examples of this monomer (second monomer component) include hydroxyethyl (meth)acrylate esters, hydroxypropyl (meth)acrylate esters, (meth)acrylic acid, itaconic acid, maleic acid, maleic anhydride, mesaconic acid, citraconic acid, glutaconic acid and the like. Of these, acrylic acid, methacrylic acid or a hydroxyethyl acrylate ester (particularly 2-hydroxyethyl acrylate) is preferred from the standpoint of availability, and acrylic acid is particularly desirable. One such monomer (second monomer component) may be used, or two or more may be used in combination.

The aforementioned other monomer (third monomer component) is used primarily to adjust the cohesiveness of the pressure-sensitive adhesive layer and to adjust the solubility or release properties of the donepezil and the like. Examples of this monomer (third monomer component) include vinyl acetate, vinyl propionate and other vinyl esters; methyl vinyl ether, ethyl vinyl ether and other vinyl ethers; N-vinyl-2-pyrrolidone, N-vinyl caprolactam and other vinyl amides; methoxyethyl (meth)acrylate ester, ethoxyethyl (meth)acrylate ester, tetrahydrofurfuryl (meth)acrylate ester and other alkoxy (meth)acrylate esters; hydroxypropyl (meth)acrylate, α-hydroxymethyl acrylate and other hydroxyl-containing monomers (which do not provide crosslinking points because they are used as the third monomer component); (meth)acrylamide, dimethyl (meth)acrylamide, N-butyl (meth)acrylamide, N-methylol (meth)acrylamide and other (meth)acrylic acid derivatives with amide groups; aminoethyl (meth)acrylate ester, dimethylaminoethyl (meth)acrylate ester, t-butylaminoethyl (meth)acrylate ester and other aminoalkyl (meth)acrylate esters; methoxyethylene glycol (meth)acrylate ester, methoxydiethylene glycol (meth)acrylate ester, methoxypolyethylene glycol (meth)acrylate ester, methoxypolypropylene glycol (meth)acrylate ester and other alkoxyalkylene glycol (meth)acrylate esters; (meth)acrylonitrile; styrene sulfonic acid, allyl sulfonic acid, sulfopropyl (meth)acrylate, (meth)acryloyl oxynaphthalene sulfonic acid, acrylamide methyl sulfonic acid and other monomers having sulfonic acid; and vinyl piperidone, vinyl pyrimidine, vinyl piperazine, vinyl pyrrole, vinyl imidazole, vinyl oxazole, vinyl morpholine and other vinyl group-containing monomers and the like. Of these, a vinyl ester or vinyl amide is preferred, and vinyl acetate is preferred as a vinyl ester, while N-vinyl-2-pyrrolidone is preferred as a vinyl amide. One such monomer (third monomer component) may be used or two or more may be used in combination.

When this acrylic pressure-sensitive adhesive is a copolymer of a (meth)acrylic acid alkyl ester (first monomer component) and a vinyl monomer having functional groups capable of contributing to a crosslinking reaction (second monomer component), the (meth)acrylic acid alkyl ester and the vinyl monomer having functional groups capable of contributing to a crosslinking reaction are preferably blended and copolymerized at a weight ratio of 99 to 85 parts of (meth)acrylic acid alkyl ester per 1 to 15 parts of vinyl monomer having functional groups capable of contributing to a crosslinking reaction, or more preferably at a weight ratio of 99 to 90 parts per 1 to 10 parts.

When the acrylic pressure-sensitive adhesive is a copolymer of a (meth)acrylic acid alkyl ester (first monomer component), a vinyl monomer having functional groups capable of contributing to a crosslinking reaction (second monomer component) and another monomer (third monomer component), the (meth)acrylic acid alkyl ester, vinyl monomer having functional groups capable of contributing to a crosslinking reaction and other monomer are preferably blended and copolymerized at a weight ratio of 40 to 94 parts of (meth)acrylic acid alkyl ester per 1 to 15 parts of vinyl monomer and 5 to 50 parts of other monomer, or more preferably at a weight ratio of 50 to 89 parts per 1 to 10 parts and 10 to 40 parts, respectively.

The polymerization reaction can be accomplished by known methods without any particular limitations, but one example is a method in which a polymerization initiator (such as benzoyl peroxide, azobisisobutyronitrile or the like) is added to the aforementioned monomers, which are then reacted for 5 to 48 hours at 50 to 70° C. in a solvent (such as ethyl acetate).

Particularly desirable acrylic pressure-sensitive adhesives in the present invention include 2-ethylhexyl acrylate ester/acrylic acid/N-vinyl-2-pyrrolidone copolymer, 2-ethylhexyl acrylate ester/2-hydroxyethyl acrylate ester/vinyl acetate copolymer, 2-ethylhexyl acrylate ester/acrylic acid copolymer and the like for example, and 2-ethylhexyl acrylate ester/acrylic acid/N-vinyl-2-pyrrolidone copolymer is especially desirable.

The glass transition temperature of the acrylic pressure-sensitive adhesive in the present invention differs depending on the copolymer composition, but from the standpoint of adhesiveness of the patch preparation it should normally be −100 to −10° C., or preferably −90° C. to −20° C.

In the patch preparation of the present invention, a liquid component may be included in the pressure-sensitive adhesive layer in order to contribute softness to the pressure-sensitive adhesive layer and reduce the pain and skin irritation caused by skin adhesiveness when the patch preparation is peeled off the skin. An organic liquid component is preferable from the standpoint of compatibility with the pressure-sensitive adhesive layer. In a patch preparation of the present invention containing an organic liquid component, depending on the kind of organic liquid component it may detract from the stability of the donepezil due for example to a chemical reaction with the donepezil. Therefore, the present invention is particularly advantageous in the case of a patch preparation containing an organic liquid component because it is capable of effectively controlling such decline of stability.

This organic liquid component may be any that is liquid at room temperature, exhibits a plasticizing effect and is compatible with the pressure-sensitive adhesive polymers making up the pressure-sensitive adhesive, without limitation, but is preferably one that improves the percutaneous absorbability and storage stability of donepezil. It can also be blended with the aim of further improving the solubility and the like of the donepezil in the pressure-sensitive adhesive. Examples of such liquid organic components include fatty acid alkyl esters (for example, esters obtained by reacting lower monohydric alcohols having 1 to 4 carbon atoms with saturated or unsaturated fatty acids having 12 to 16 carbon atoms); saturated or unsaturated fatty acids having 8 to 10 carbon atoms (for example, caprylic acid (octanoic acid, C8), pelargonic acid (nonanoic acid, C9), capric acid (decanoic acid, C10), lauric acid (C12), etc.); ethylene glycol, diethylene glycol, triethylene glycol, polyethylene glycol, propylene glycol, polypropylene glycol and other glycols; olive oil, castor oil, squalene, lanoline and other oils and fats; ethyl acetate, ethyl alcohol, dimethyl decyl sulfoxide, decyl methyl sulfoxide, dimethyl sulfoxide, dimethyl formamide, dimethyl acetamide, dimethyl lauryl amide, dodecyl pyrrolidine, isosorbitol, oleyl alcohol and other organic solvents; liquid surfactants; diisopropyl adipate, phthalic acid esters, diethyl sebacate and other plasticizers; and liquid paraffin and other hydrocarbons and the like. Other examples include ethoxylated stearyl alcohol, glycerin esters (those liquid at room temperature), isotridecyl myristate, N-methylpyrrolidone, ethyl oleate, oleic acid, diisopropyl adipate, octyl palmitate, 1,3-propanediol, glycerin and the like. Of these, a fatty acid alkyl ester, saturated fatty acid, hydrocarbon or organic solvent is preferred from the standpoint of stability of the preparation and the like, and a fatty acid alkyl ester is especially preferred. One such liquid organic component may be used alone or two or more may be used in combination.

Of these, when an acrylic pressure-sensitive adhesive is used for the pressure-sensitive adhesive the organic liquid component should be a fatty acid alkyl ester for purposes of compatibility and the like with the acrylic pressure-sensitive adhesive, and should more preferably be an ester obtained by reacting a lower monohydric alcohol having 1 to 4 carbon atoms with a saturated or unsaturated fatty acid having 12 to 16 carbon atoms. This saturated or unsaturated fatty acid having 12 to 16 carbon atoms is preferably a saturated fatty acid, and the lower monohydric alcohol having 1 to 4 carbon atoms may be either straight-chain or branched. Desirable examples of fatty acids having 12 to 16 carbon atoms include lauric acid (C12), myristic acid (C14), palmitic acid (C16) and the like, while desirable examples of lower monohydric alcohols having 1 to 4 carbon atoms include isopropyl alcohol, ethyl alcohol, methyl alcohol, propyl alcohol and the like. Specific examples of particularly desirable fatty acid alkyl esters include isopropyl myristate, ethyl laurate, isopropyl palmitate and the like.

When using a fatty acid alkyl ester, a fatty acid having 8 to 10 carbon atoms and/or glycerin may be used in combination with the fatty acid alkyl ester in order to improve the percutaneous absorbability of the donepezil.

The blended amount of the organic liquid component in the present invention is preferably 10 to 160 parts by weight or more preferably 40 to 150 parts by weight based on 100 parts by weight of the pressure-sensitive adhesive. If the blended amount is less than 10 parts by weight, the pressure-sensitive adhesive layer may not be sufficiently plasticized to produce the desired softness or reduce skin irritancy sufficiently, while if the amount exceeds 160 parts by weight, it is likely that the organic liquid component will not be retained in the pressure-sensitive adhesive even by the cohesive force of the adhesive, resulting in blooming on the surface of the pressure-sensitive adhesive layer and weakening the adhesive force to the point that the preparation may become detached from the skin surface during use.

In the patch preparation of the present invention, the pressure-sensitive adhesive layer may be crosslinked by a known chemical crosslinking process (using a crosslinking agent or the like) or physical crosslinking process (exposure to ultraviolet rays or gamma rays or other electron rays or the like) as discussed above, and this crosslinking process may be one commonly used in the technical field. In the patch preparation of the present invention, the stability of the donepezil may decline during manufacture or storage of the preparation depending on the chemical or physical crosslinking process used (namely, more related substances may be produced). Consequently, the present invention is particularly useful when applied to a patch preparation in which the pressure-sensitive adhesive layer has been crosslinked. A chemical crosslinking process using a crosslinking agent is desirable because it is less likely to adversely affect the donepezil.

In the case of a chemical crosslinking process using a crosslinking agent, the crosslinking agent is not particularly limited as long as crosslink formation is not impeded by the donepezil, and examples include peroxides (such as benzoyl peroxide (BPO) and the like), metal oxides (such as magnesium metasilicate aluminate and the like), polyfunctional isocyanate compounds, organic metal compounds (such as zirconium alaninate, zinc alaninate, zinc acetate, glycine ammonium zinc, titanium compounds and the like), metal alcoholates (such as tetraethyl titanate, tetraisopropyl titanate, aluminum isopropylate, aluminum sec-butyrate and the like) and metal chelate compounds (such as dipropoxy bis(acetylacetonate) titanium, tetraoctylene glycol titanium, aluminum isopropylate, ethylacetoacetate aluminum diisopropylate, aluminum tris(ethylacetoacetate) and aluminum tris(acetylacetonate) and the like. Of these, a peroxide, metal oxide, organic metal compound, metal alcoholate or metal chelate compound is preferred and a metal alcoholate or metal chelate compound is more preferred from the standpoint of efficiently forming crosslinks in the presence of donepezil, while a metal chelate compound is best for easily obtaining crosslinked structures with a suitable crosslinking density. Of the metal chelate compounds, ethylacetoacetate aluminum diisopropylate is particularly desirable. One such crosslinking agent may be used or two or more may be used in combination.

The blended amount of the crosslinking agent differs depending on the type of crosslinking agent and pressure-sensitive adhesive, but is normally 0.1 to 0.6 parts by weight or preferably 0.15 to 0.5 parts by weight based on 100 parts by weight of the pressure-sensitive adhesive. Below 0.1 parts by weight the crosslinking points are too few to contribute sufficient cohesiveness to the pressure-sensitive adhesive layer, posing a risk of adhesive residue and strong skin irritancy due to cohesive failure during peeling, while above 0.6 parts by weight there is more cohesiveness but sufficient skin adhesiveness may not be obtained. Moreover, there may be skin irritation caused by a residue of unreacted crosslinking agent.

Chemical crosslinking treatment can be accomplished for example by first adding the crosslinking agent followed by a step of heating and storing at or above the crosslinking reaction temperature, or in other words a curing step, and the heating temperature in this case is selected appropriately according to the type of crosslinking agent but is preferably 60 to 90° C. or more preferably 60 to 80° C. The heating time is preferably 12 to 96 hours or more preferably 24 to 72 hours.

In the patch preparation of the present invention, a metal chloride can be included together with donepezil in the crosslinked pressure-sensitive adhesive layer. When a metal chloride is included in the pressure-sensitive adhesive layer there is less decline in cohesiveness of the pressure-sensitive adhesive layer while the patch preparation is attached to human skin, and less risk of cohesive failure when the pressure-sensitive adhesive layer is peeled off.

This metal chloride is not particularly limited but may be a chloride of an alkaline metal such as sodium or potassium; a chloride of an alkaline earth metal such as calcium or magnesium; or aluminum chloride, stannous chloride, ferric chloride or the like. From the standpoint of stability and controlling decline of cohesiveness of the pressure-sensitive adhesive layer, sodium chloride, calcium chloride, aluminum chloride, stannous chloride or ferric chloride is preferred, sodium chloride or calcium chloride is more preferred, and sodium chloride is especially preferred. Any of these may be used alone or two or more may be used in combination. The blended amount of the metal chloride is preferably 0.1 to 20 parts by weight or more preferably 1 to 15 parts by weight or most preferably 3 to 10 parts by weight based on 100 parts by weight of the pressure-sensitive adhesive. If the blended amount is less than 0.1 parts by weight it may be insufficient to control the decline in cohesiveness of the pressure-sensitive adhesive layer, while if it exceeds 20 parts by weight, it will have a controlling effect but will not be uniformly dispersed in the pressure-sensitive adhesive (pressure-sensitive adhesive polymer), potentially detracting from the appearance of the preparation.

In the present invention, the metal chloride may be one produced when donepezil hydrochloride is neutralized with an inorganic base containing a metal in the process of forming the pressure-sensitive adhesive layer, and examples of such inorganic bases containing metals include sodium hydroxide, potassium hydroxide, calcium hydroxide, magnesium hydroxide, sodium hydrogencarbonate, potassium hydrogencarbonate, sodium carbonate, potassium carbonate and other inorganic bases of alkaline metals or alkaline earth metals or the like, but a hydroxide of an alkaline metal or alkaline earth metal is preferred from the standpoint of avoiding by-products, and sodium hydroxide, calcium hydroxide and magnesium hydroxide are especially preferred, with sodium hydroxide being most desirable.

In the patch preparation of the present invention, the thickness of the pressure-sensitive adhesive layer is preferably 20 to 300 μm or more preferably 30 to 300 μm or still more preferably 50 to 300 μm. If the pressure-sensitive adhesive layer is less than 20 μm thick it will be difficult to obtain sufficient adhesive force or to include an effective dose of donepezil, while if it is more than 300 μm thick the coating process may be difficult.

The patch preparation of the present invention includes a support and a pressure-sensitive adhesive layer, and preferably has a release liner. Namely, the patch preparation of the present invention has a structure wherein the pressure-sensitive adhesive layer described above is laminated on at least one side of a support, and the adhesive surface of the pressure-sensitive adhesive layer (the surface opposite the side laminated on the support) is preferably protected by being covered with a release liner until immediately before use. The preparation may also be made into a roll without the use of a release liner if the support is coated with a silicone, fluorine, wax or other backing agent.

The support is not particularly limited but is preferably one that does not allow the donepezil in the pressure-sensitive adhesive layer to pass through the support and be lost through the back, reducing the content of the drug (namely, a material that is impermeable to donepezil), and is also preferably one that does not allow the donepezil and organic liquid component to pass through the support and be lost from the back when an organic liquid component is contained in the pressure-sensitive adhesive layer as discussed below, reducing the content of the drug (namely, a material that is impermeable to the organic liquid component and donepezil).

Specific examples include polyester (such as polyethylene terephthalate (PET) or the like), nylon, polyvinyl acetate, polyethylene, polypropylene, ethylene-vinyl acetate copolymer, polytetrafluoroethylene, ionomer resins and other single films, metal foil, and laminate films obtained by laminating two or more such films. Of these, the support is preferably a laminate film obtained by laminating a nonporous film consisting of one of the aforementioned materials with a porous film as described below in order to improve the adhesiveness (anchoring properties) of the support with the pressure-sensitive adhesive layer, with the pressure-sensitive adhesive layer preferably being formed on the porous film side.

This porous film is not particularly limited as long as it improves anchorage properties with the pressure-sensitive adhesive layer, and examples include paper, woven fabrics, nonwoven fabrics (such as polyester (for example, polyethylene terephthalate (PET)) nonwoven fabric and the like), and films obtained by mechanical perforation of the aforementioned films (such as polyester, nylon, Saran™, polyethylene, polypropylene, ethylene-vinyl acetate copolymer, polyvinyl acetate, ethylene-ethyl acrylate copolymer, polytetrafluoroethylene, metal foil, polyethylene terephthalate and other single films and laminate films obtained by laminating one or two or more such films), and paper, nonwoven fabrics and woven fabrics (for example, polyester nonwoven fabric, polyethylene terephthalate nonwoven fabric and the like) are preferred from the standpoint of flexibility of the support. For purposes of improved anchorage properties and flexibility of the pressure-sensitive adhesive layer, the thickness of the porous film is normally 10 to 500 μm, and in the case of a thin patch preparation such as a plaster or adhesive tape, it is normally about 1 to 200 μm. In the case of woven fabrics and nonwoven fabrics, the basis weight should preferably be 5 to 30 g/m² for purposes of improving anchorage properties.

The thickness of the support for the patch preparation of the present invention is not particularly limited but is preferably 2 to 200 μm or more preferably 10 to 50 μm. Below 2 μm the handling properties such as self-supporting properties may be adversely affected, while above 200 μm the support may feel unpleasant (stiff), detracting from compliance.

The release liner is not particularly limited, and a known release liner may be used. Specifically, the release liner may be a release liner comprising a release agent layer of a release agent formed on the surface of a release sheet base, or a plastic film which itself has good release properties, or a release liner comprising a release layer of such a plastic film material with good release properties formed on the surface of a release sheet base. The release liner may have a release surface on only one side of the base, or on both sides.

The release agent in this release liner is not particularly limited, and examples include long-chain alkyl group-containing polymers, silicone polymers (silicon release agents), fluorine polymers (fluorine release agents) and other release agents. The base for the release liner may be of polyethylene terephthalate (PET) film, polyimide film, polypropylene film, polyethylene film, polycarbonate film, polyester (other than PET) film or other plastic film, or of metal-deposited plastic film obtained by depositing a metal on one of these films, or of paper, Japanese paper, kraft paper, glassine paper, fine paper (bookpaper) or other paper, or of nonwoven fabric, fabric or other fibrous material, or of metal foil or the like.

Examples of plastic films that themselves have good release properties include polyethylene (low-density polyethylene, linear low-density polyethylene, etc.), polypropylene, ethylene-propylene copolymer and other ethylene-α-olefin copolymers (block copolymers or random copolymers), as well as polyolefin films formed from polyolefin resins consisting of mixtures of these, and Teflon films and the like for example.

The release layer formed on the surface of the aforementioned release liner base can be formed by laminating or coating the material of the aforementioned plastic film with good release properties on the aforementioned release liner base.

The thickness (overall thickness) of the release liner is not particularly limited but is normally 200 μm or less or preferably 25 to 100 μm.

In the present invention the method for preparing the patch preparation is not particularly limited, but in one embodiment it is prepared by forming a film of a mixture containing at least a pressure-sensitive adhesive, donepezil and a stabilizer to thereby form the pressure-sensitive adhesive layer containing donepezil. Namely, the present invention also relates to a method for preparing a patch preparation containing donepezil, wherein a pressure-sensitive adhesive layer containing donepezil is formed on at least one side of a support by forming a film of a mixture containing a pressure-sensitive adhesive, donepezil and the aforementioned stabilizer. It also relates to a method for stabilizing donepezil in a patch preparation that comprises including the aforementioned stabilizer with donepezil in the presence of a pressure-sensitive adhesive.

In this embodiment, because the stabilizer is applied to the donepezil in the mixture for forming the pressure-sensitive adhesive layer, the donepezil is stabilized from the time the mixture is prepared for forming the pressure-sensitive adhesive layer, and related substances can thus be adequately controlled.

Specifically, in one method a pressure-sensitive adhesive, donepezil and a stabilizer or the like for example are dissolved or dispersed in a solvent and mixed, the resulting solution or dispersion is coated on at least one side of a support and dried to form a film as the pressure-sensitive adhesive layer on the surface of the support, and a release liner is then applied. Alternatively, for example the aforementioned solution or dispersion can be applied to at least one side of a protective release liner and dried to form a film as the pressure-sensitive adhesive layer on the surface of the release liner, and the support can then be affixed to the pressure-sensitive adhesive layer to prepare the patch.

The solvent for dissolving or dispersing the pressure-sensitive adhesive and the like may be ethyl acetate, toluene, hexane, 2-propanol, methanol, ethanol, water or the like for example. These may also be used to adjust the viscosity after addition of the crosslinking agent.

When the pressure-sensitive adhesive layer is to be crosslinked, the crosslinking agent is preferably added to the aforementioned solution or dispersion for purposes of chemical crosslinking treatment. Also, when the pressure-sensitive adhesive layer is to be crosslinked the pressure-sensitive adhesive layer is preferably stored (aging step) after film formation in order to promote crosslinking. This aging step is normally accomplished after the solution or dispersion has been coated and dried to form a film (after formation of the pressure-sensitive adhesive layer) by leaving the resulting pressure-sensitive adhesive layer for about 12 to 96 hours (preferably about 24 to 72 hours) with heating at 60 to 90° C. (preferably 60 to 80° C.). The donepezil can also be stabilized during this period because it is together with a stabilizing agent, thereby suppressing production of related substances.

The patch preparation of the present invention is not particularly limited as to form and may be a tape, sheet, matrix, reserver, controlled release film or the like. The stabilization effect of the present invention is useful for tapes and sheets because these are vulnerable to the effects of the environment and particularly of oxygen.

The dosage of the patch preparation of the present invention differs depending on the types and amounts of pressure-sensitive adhesive and organic liquid component used and on the age, weight and symptoms of the patient and the like, but in the case of an adult a patch preparation containing 2 to 150 mg of donepezil or donepezil hydrochloride should normally be administered to a 5 to 120 cm² patch of skin for 1 to 7 days.

### EXAMPLES

The present invention is explained in more detail below using examples. Unless otherwise specified, “parts” in the description means parts by weight.

### Examples 1 to 19 and Comparative Examples 1 to 6

75 parts of 2-ethylhexyl acrylate, 22 parts of N-vinyl-2-pyrrolidone, 3 parts of acrylic acid and 0.2 parts of azobisisobutyronitrile were solution polymerized in ethyl acetate at 60° C. in an inactive gas atmosphere to obtain an ethyl acetate solution of pressure-sensitive adhesive A (pressure-sensitive adhesive solids: 28%).

Next, coating solutions were obtained containing the stabilizers shown in Table 2 below, mixed with the composition of Table 1 below and with the viscosity adjusted with ethyl acetate. These were applied to a dried thickness of 60 μm to polyethylene terephthalate (PET) release liners and dried, and PET supports were affixed thereto to obtain patch preparations which were then stored for 48 hours at 70° C. to obtain the stored patch preparations of Examples 1 to 19 and Comparative Examples 1 to 6.

TABLE 1

| | |
|---|---|
| Pressure-sensitive adhesive A | 40 parts |
| Isopropyl myristate | 50 parts |
| Donepezil hydrochloride | 8.3 parts |
| Sodium hydroxide | 0.8 parts |
| Ethyl acetoacetate aluminum diisopropylate | 0.2 parts |
| Stabilizer | 1.0 part or 0 parts |
| | (Comparative |
| | Example 1) |

(Measuring percentage of related substances)

The patch preparations of the Examples and Comparative Examples were extracted with methanol, and these extracts were analyzed by HPLC under the following conditions.

### (HPLC Conditions)

HPLC column: Inertsil™ ODS-2 (4.6 mm I.D.×15 cm, 5 μm) GL Science

Column temperature: 35° C.

Mobile phase: Sodium 1-decansulfonate aqueous solution/acetonitrile/70% perchloric acid=650/350/1 (volume ratio)

Sodium 1-decansulfonate concentration: 10 mM of total mobile phase

Flow rate: 1.4 mL/min

Detection: UV (271 nm)

Retention time Donepezil=11.0 min., related substance 1=12.8 min., related substance 2=3.9 min.

The area ratios of the areas of the HPLC peaks for donepezil-related substances 1 and 2 and total related substances to the area of the HPLC peak for donepezil in the pressure-sensitive adhesive layers of the stored patch preparations of Examples 1 to 19 and Comparative Examples 1 to 6 are shown in Table 2 as the percentage of donepezil-related substance 1, the percentage of donepezil-related substance 2 and the percentage of all donepezil-related substances, respectively.

TABLE 2

| | | | | % total |
|---|---|---|---|---|
| | | % | % related | related |
| | Stabilizer | related substance 1 | substance 2 | substances |
| Ex 1 | L(+)-ascorbic acid | 0.5% | 0.0% | 0.6% |
| Ex 2 | L-ascorbyl palmitate | 0.3% | 0.1% | 0.5% |
| Ex 3 | D(−)-isoascorbic acid | 0.3% | 0.0% | 0.5% |
| Ex 4 | Sodium isoascorbate | 0.5% | 0.1% | 0.9% |
| | monohydrate | | | |
| Ex 5 | Ethylenediamine-N,N,N′N′- | 0.7% | 0.1% | 1.1% |
| | calcium tetraacetate (II) | | | |
| | disodium salt dihydrate | | | |
| Ex 6 | L-cysteine | 0.2% | 1.3% | 4.5% |
| Ex 7 | 2-mercaptobenzimidazole | nd | 0.0% | 0.6% |
| Ex 8 | 3(2)-t-butyl-4- | 3.4% | 0.1% | 6.7% |
| | hydroxyanisole | | | |
| Ex 9 | 2,6-di-t-butyl-4- | 0.4% | 0.0% | 0.7% |
| | methylphenol | | | |
| Ex 10 | Tetrakis[3-(3′,5′-di-t-butyl-4′- | 0.6% | 0.0% | 1.0% |
| | hydroxyphenyl)propionic | | | |
| | acid]pentaerythritol | | | |
| Ex 11 | 3-mercapto-1,2-propanediol | 0.7% | 2.5% | 5.5% |
| Ex 12 | Acetic acid(±)-α-tocopherol | 1.0% | 0.1% | 1.5% |
| Ex 13 | Rutin | 0.3% | 0.0% | 0.5% |
| Ex 14 | Quercetin dihydrate | 0.3% | 0.0% | 16.1% |
| Ex 15 | Hydroquinone | 5.0% | 0.1% | 9.5% |
| Ex 16 | Sodium hydroxymethane- | nd | 0.8% | 3.8% |
| | sulfinate dihydrate | | | |
| Ex 17 | Sodium metabisulfite | nd | 1.0% | 3.6% |
| Ex 18 | Sodium sulfite | 0.1% | 0.4% | 1.3% |
| Ex 19 | Sodium thiosulfate | nd | 0.1% | 0.4% |
| CE 1 | None | 1.2% | 0.2% | 1.9% |
| CE 2 | Propyl gallate | 2.1% | 0.2% | 4.2% |
| CE 3 | 1,3-butanediol | 1.4% | 0.2% | 2.2% |
| CE 4 | (±)-α-tocopherol | 27.0% | 1.1% | 67.9% |
| CE 5 | 1H-benzotriazole | 1.4% | 0.3% | 2.1% |
| CE 6 | Hypophosphorous acid | 1.3% | 0.2% | 1.9% |

Note)
nd in table means “not detected”

As shown in Table 2, in comparison with Comparative Example 1 the percentage of either related substance 1, related substance 2 or total related substances was improved in Examples 1 to 19, which had specific stabilizers blended therein. Namely, the percentage of related substance 1 was lower in Examples 1 to 7, 9 to 14 and 16 to 19. The percentage of related substance 2 was lower in Examples 1 to 5, 7 to 10, 12 to 15 and 19. Moreover, the percentage of total related substances was lower in Examples 1 to 5, 7, 9 to 10, 12 to 13 and 18 to 19.

In Comparative Examples 2 to 6, no suppressive effect on production of related substance was seen despite the addition of ordinary stabilizers, and in fact the percentage of either related substance 1 or related substance 2 was actually greater in all cases than in Comparative Example 1 having no added stabilizer, while the percentage of total related substances was greater in all cases. In particular, in Comparative Example 4 using (±)-α-tocopherol about 22.5 times the amount of related substance 1 and about 35.7 times the amount of total related substances was produced as in Comparative Example 1 having no added stabilizer. This shows that ordinary stabilizers may actual impede stabilization of a donepezil-containing patch preparation.

### Examples 20 to 24

The stored patch preparation of Example 20 was obtained as in Example 1 except that the 1.0 part of L(+)-ascorbic acid (hereinafter “ascorbic acid”) used in Example 1 was replaced with 0.5 parts of ascorbic acid and 0.5 parts of D(−)-isoascorbic acid (hereinafter called “isoascorbic acid”). The stored patch preparations of Examples 21 through 24 were likewise obtained as in Example 20 except that the 0.5 parts of ascorbic acid and 0.5 parts of isoascorbic acid used in Example 20 were replaced with the combinations of stabilizers shown in Table 3 (0.5 parts each). These patch preparations were analyzed as described above.

TABLE 3

| | | | | % total |
|---|---|---|---|---|
| | | % related | % related | related |
| | Stabilizer | substance 1 | substance 2 | substances |
| Ex | L(+)-ascorbic acid/D(−)- | 0.6% | 0.1% | 0.8% |
| 20 | isoascorbic acid | (0.40%) | (0%) | (0.55%) |
| Ex | L(+)-ascorbic acid/2- | 0.0% | nd | 0.1% |
| 21 | mercaptobenzimidazole | (0.25%) | (0%) | (0.60%) |
| Ex | L(+)-ascorbic acid/2,6-di-t- | 0.4% | 0.1% | 0.8% |
| 22 | butyl-4-methylphenol | (0.45%) | (0%) | (0.65%) |
| Ex | L(+)-ascorbic acid/sodium | Nd | 0.0% | 0.7% |
| 23 | hydroxymethane sulfinate | (0.25%) | (0.40%) | (2.2%) |
| | dihydrate | | | |
| Ex | L(+)-ascorbic acid/rutin | 0.7% | 0.1% | 1.1% |
| 24 | | (0.40%) | (0%) | (0.60%) |

Note 1)
nd in table means “not detected”
Note 2)
In the columns for % of related substance 1, % of related substance 2 and % of total related substances, the numbers in parenthesis indicate the arithmetic mean of the percentages of related substances in Table 3 for the stabilizers combined in each example. When calculating the arithmetic mean, nd in Table 3 was given as 0%.

As shown in Table 3, at least one of the percentage of related substance 1, the percentage of related substance 2 and the percentage of total related substances was lower in Examples 20 through 24 than in Comparative Example 1 in Table 2. In particular, the percentage of related substance 1 was synergistically reduced in Examples 21 through 23, and in Example 21 not only the percentage of related substance 1 but also the percentage of total related substances was synergistically reduced, while in Example 23 not only the percentages of related substance 1 and related substance 2 but also the percentage of total related substances was synergistically reduced.

### Examples A through E

The stored patch preparation of Example A was obtained as in Example 1 except that the 1.0 part of ascorbic acid used in Example 1 was replaced with 0.99 parts of ascorbic acid and 0.01 part of sodium metabisulfite. Similarly to Example A, the stored patch preparations of Examples B through I were obtained with the blended amounts of these stabilizers varied as shown in Table 4.

These patch preparations were analyzed as described above. The adhesive properties of the pressure-sensitive adhesive layer were evaluated functionally according to the following standard:

[symbol] Extremely good adhesive properties

◯ Good adhesive properties

A Adhesive properties somewhat poor but within acceptable range

### The results are shown in Table 4.

TABLE 4

| | L(+)-ascorbic | | | % total | |
|---|---|---|---|---|---|
| | acid*/sodium | % related | % related | related | Adhesive |
| | metabisulfite | substance 1 | substance 2 | substances | properties |
| Ex A | 0.99 (9.99%)/0.01 | 0.1% | 0.0% | 0.2% | Δ |
| | (100:1) | | | | |
| Ex B | 0.91 (0.91%)/0.09 (10:1) | 0.1% | 0.0% | 0.3% | Δ* |
| Ex C | 0.50 (0.50%)/0.50 (1:1) | 0.0% | 0.0% | 0.2% | ◯ |
| Ex D | 0.09 (0.09%)/0.91 (1:10) | nd | 0.0% | 0.2% | [symbol] |
| Ex E | 0.01 (0.01%)/0.99 | nd | 0.4% | 2.1% | [symbol] |
| | (1:100) | | | | |
| Ex 17 | 0 parts (0%)/1 part | nd | 1.0% | 3.6% | [symbol] |
| (described | | | | | |
| above) | | | | | |
| Ex F | 0.50 (0.50%)/0.50 (1:1) | 0.0% | 0.0% | 0.2% | ◯ |
| Ex G | 0.10 (0.10%)/0.50 (1:5) | nd | 0.0% | 0.2% | ( |
| Ex H | 0.05 (0.05%)/0.50 (1:10) | nd | 0.0% | 0.2% | ( |
| Ex I | 0.02 (0.02%)/0.50 (1:25) | nd | 0.0% | 0.4% | ( |
| Ref | Arithmetic mean of Ex 1 | 0.25% | 0.5% | 2.1% | — |
| | and Ex 17 (see above) | | | | |

Note 1)
% values in parentheses represent wt % of ascorbic acid based on the total weight of the pressure-sensitive adhesive layer
Note 2)
(The adhesive properties were better in Example B than in Example A The results of Example 1 showed that ascorbic acid particularly suppresses production of related substance 2.

The results of Table 4 show that production of related substance 2 was suppressed in Examples A through D. However, when the percentage of ascorbic acid was reduced to 0.01 wt % based on the total weight of the pressure-sensitive adhesive layer, production of related substance 2 increased. Looking at Table 4, production of related substance 2 was suppressed when the content of ascorbic acid was 0.02 wt % based on the total weight of the pressure-sensitive adhesive layer. Table 4 also shows that the adhesive properties declined somewhat when ascorbic acid was included in amounts of close to 1 wt % based on the total weight of the pressure-sensitive adhesive layer but were better when 0.5 wt % was included, and even better when 0.1 wt % was included. Since the stabilizing effect was greater in Example E than in Example 17, and since the adhesive properties were better in Example C than in Example B and better in Example B than in Example A, the weight ratio of ascorbic acid to sodium metabisulfite is preferably 100:1 to 1:100 or more preferably 10:1 to 1:100 or still more preferably 1:1 to 1:100. Moreover, the produced amounts of each component were dramatically suppressed in Example F in comparison with the Comparative Examples, indicating a synergistic stabilizing effect.

Furthermore, the results of Table 4 show that the stabilizing effect was clearly confirmed when ascorbic acid and sodium metabisulfite was included in a range of 0.01 to 9.99 wt % and 0.01 to 9.99 wt % based on the total weight of the pressure-sensitive adhesive layer (namely, the total solids weight of the mixture used to form the pressure-sensitive adhesive layer), or more preferably in a range of 0.02 to 9.99 wt % and 0.01 to 9.91 wt %. Moreover, the results of Table 4 show that the weight ratio of ascorbic acid to sodium metabisulfite is preferably 1:100 to 100:1 or more preferably 1:10 to 100:1 or still more preferably 1:25 to 100:1 from the standpoint of stability.

### Examples 25 to 34

75 parts of 2-ethylhexyl acrylate, 22 parts of N-vinyl-2-pyrrolidone, 3 parts of acrylic acid and 0.2 parts of azobisisobutyronitrile were solution polymerized in ethyl acetate at 60° C. in an inactive gas atmosphere to obtain an ethyl acetate solution of pressure-sensitive adhesive A (pressure-sensitive adhesive solids: 28%). Next, coating solutions were obtained containing 0.5 parts of respective stabilizers shown in Table 6 below, mixed with the composition of Table 5 below and with the viscosity adjusted with ethyl acetate. These were applied to a dried thickness of 60 μm to polyethylene terephthalate (PET) release liners and dried, and PET supports were affixed thereto to obtain patch preparations which were then stored for 48 hours at 70° C. to obtain the stored patch preparations of Examples 25 to 34.

TABLE 5

| | |
|---|---|
| Pressure-sensitive adhesive A | 40 parts |
| Isopropyl myristate | 50 parts |
| Donepezil hydrochloride | 8.3 parts |
| Sodium hydroxide | 0.8 parts |
| Ethyl acetoacetate aluminum diisopropylate | 0.2 parts |
| Stabilizer 1 | 0.5 parts |
| Stabilizer 2 | 0.5 parts |

### Comparative Example 7

The stored patch preparation of Comparative Example 7 was obtained as in Example 25 except that the 0.5 parts of D(−)-isoascorbic acid (hereinafter “isoascorbic acid”) and 0.5 parts of 2-mercaptobenzimidazole added in Example 25 were not added.

### Reference Examples 1 to 5

The stored patch preparation of Reference Example 1 was obtained as in Example 25 except that the 0.5 parts of isoascorbic acid and 0.5 parts of 2-mercaptobenzimidazole added in Example 25 were replaced with 1 part of isoascorbic acid. The patch preparations of Reference Examples 2 through 5 were also obtained as in Reference Example 1 except that the 1 part of isoascorbic acid was replaced with 1 part each of the stabilizers shown in Table 6 in Reference Example 1.

### (Measuring Percentages of Related Substances)

The patch preparations of the Examples and Reference Examples were methanol extracted, and the extracts were analyzed by HPLC under the following conditions.

### (HPLC Conditions)

HPLC column: Inertsil™ ODS-2 (4.6 mm I.D.×15 cm, 5 μm), GL Science

Column temperature: 35° C.

Mobile phase: sodium 1-decansulfonate aqueous solution/acetonitrile/70% perchloric acid=650/350/1 (volume ratio); sodium 1-decansulfonate concentration 10 mM of total mobile phase

Flow rate: 1.4 mL/min

Detection: UV (271 nm)

Retention times: Donepezil=11.0 min, related substance 1=12.8 min, related substance 2=3.9 min

The area ratios of the areas of the HPLC peaks for donepezil-related substances 1 and 2 and total related substances to the area of the HPLC peak for donepezil in the pressure-sensitive adhesive layers of the stored patch preparations of Examples 25 to 34, Comparative Example 7 and Reference Examples 1 to 5 are shown in Table 6 as the percentage of donepezil-related substance 1, the percentage of donepezil-related substance 2 and the percentage of all donepezil-related substances, respectively.

TABLE 6
[translator's note: Examples 25-34 are not listed in the table]

| | | | | % total |
|---|---|---|---|---|
| | | % related | % related | related |
| | Stabilizer | substance 1 | substance 2 | substances |
| Ex | D(−)-isoascorbic acid/2- | 0.0% (0.15%) | 0.0% (0%) | 0.9% (0.55%) |
| 25 | mercaptobenzimidazole | | | |
| Ex | D(−)-isoascorbic acid/2.6-di-t- | 0.6% (0.35%) | 0.1% (0%) | 1.1% (0.55%) |
| 26 | butyl-4-methylphenol | | | |
| Ex | D(−)-isoascorbic acid/sodium | nd (0.15%) | 0.2% (0.4%) | 1.0% (2.15%) |
| 27 | hydroxymethane sulfinate | | | |
| | dihydrate | | | |
| Ex | D(−)-isoascorbic acid/rutin | 0.8% (0.3%) | 0.1% (0%) | 1.0% (0.5%) |
| 28 | | | | |
| Ex | 2-mercaptobenzimidazole/2,6- | nd (0.2%) | 0.0% (0%) | 0.6% (0.65%) |
| 29 | di-t-butyl-4-methylphenol | | | |
| Ex | 2-mercaptobenzimidazole/ | nd (0%) | 0.7% (0.4%) | 3.3% (2.2%) |
| 30 | sodium hydroxymethane | | | |
| | sulfinate dihydrate | | | |
| Ex | 2-mercaptobenzimidazole/rutin | nd (0.15%) | 0.0% (0%) | 0.6% (0.55%) |
| 31 | | | | |
| Ex | 2,6-di-t-butyl-4-methylphenol/ | 0.0% (0.2%) | 0.1% (0.4%) | 0.9% (2.25%) |
| 32 | sodium hydroxymethane | | | |
| | sulfinate dihydrate | | | |
| Ex | 2,6-di-t-butyl-4- | 0.4% (0.25%) | 0.0% (0%) | 0.9% (0.6%) |
| 33 | methylphenol/rutin | | | |
| Ex | Sodium hydroxymethane | nd (0.15%) | 0.5% (0.4%) | 1.8% (2.15%) |
| 34 | sulfinate dihydrate/rutin | | | |
| CE 7 | None | 1.2% | 0.2% | 1.9% |
| RE 1 | D(−)-isoascorbic acid | 0.3% | 0.0% | 0.5% |
| RE 2 | 2-mercaptobenzimidazole | nd | 0.0% | 0.6% |
| RE 3 | 2,6-di-t-butyl-4-methylphenol | 0.4% | 0.0% | 0.7% |
| RE 4 | Sodium hydroxymethane | nd | 0.8% | 3.8% |
| | sulfinate dihydrate | | | |
| RE 5 | Rutin | 0.3% | 0.0% | 0.5% |

Note 1)
nd in table means “not detected”
Note 2)
In the examples, the numbers in parentheses in the columns for % of related substance 1, % of related substance 2 and % of total related substances indicate the arithmetic mean of the corresponding numbers in the Reference Examples. When calculating the arithmetic mean, nd in table was given as 0%.

Table 6 shows that the produced amount of related substance 1 was lower in all Examples than in Comparative Example 7. Production of total related substances was also suppressed in all Examples except Example 30. In Examples 25, 26, 28, 29, 31, 32 and 33, the produced amount of related substance 2 was lower than in Comparative Example 7.

In comparison with Reference Examples 1 to 5, the produced amount of related substance 1 was synergistically suppressed in Examples 25, 27, 29, 31, 32 and 34. The produced amount of related substance 2 was synergistically suppressed in Examples 27 and 32. The produced amount of total related substances was synergistically suppressed in Examples 27, 29, 32 and 34.

I(We) claim:

1. A patch preparation comprising, a support and a pressure-sensitive adhesive layer on at least one side of the support, the pressure-sensitive adhesive layer comprising a pressure-sensitive adhesive, donepezil and a stabilizer,
wherein said stabilizer comprises at least one or more species selected from the group consisting of ascorbic acid, metal salts or esters thereof, isoascorbic acid or metal salts thereof, ethylenediamine tetraacetic acid or metal salts thereof, cysteine, acetylcysteine, 2-mercaptobenzimidazole, 3(2)-t-butyl-4-hydroxyanisole, 2,6-di-t-butyl-4-methylphenol, tetrakis[3-(3′,5′-di-t-butyl-4′-hydroxyphenyl)propionic acid]pentaerythritol, 3-mercapto-1,2-propanediol, tocopherol acetate, rutin, quercetin, hydroquinone, metal salts of hydroxymethanesulfinic acid, metal metabisulfite salts, metal sulfite salts and metal thiosulfate salts.

2. The patch preparation according to claim 1, wherein said stabilizer comprises at least one or more species selected from the group consisting of ascorbic acid, metal salts or esters thereof, isoascorbic acid or metal salts thereof, ethylenediamine tetraacetic acid or metal salts thereof, cysteine, acetylcysteine, 2-mercaptobenzimidazole, 2,6-di-t-butyl-4-methylphenol, tetrakis[3-(3′,5′-di-t-butyl-4′-hydroxyphenyl)propionic acid]pentaerythritol, 3-mercapto-1,2-propanediol, tocopherol acetate, rutin, quercetin, metal salts of hydroxymethanesulfinic acid, metal metabisulfite salts, metal sulfite salts and metal thiosulfate salts.

3. The patch preparation according to claim 1, wherein said stabilizer comprises at least one or more species selected from the group consisting of ascorbic acid, metal salts or esters thereof, isoascorbic acid or metal salts thereof, ethylenediamine tetraacetic acid or metal salts thereof, 2-mercaptobenzimidazole, 2,6-di-t-butyl-4-methylphenol, tetrakis[3-(3′,5′-di-t-butyl-4′-hydroxyphenyl)propionic acid]pentaerythritol, tocopherol acetate, rutin, metal sulfite salts and metal thiosulfate salts.

4. The patch preparation according to claim 1, wherein said stabilizer comprises at least one or more species selected from the group consisting of ascorbic acid, metal salts or esters thereof, isoascorbic acid or metal salts thereof, ethylenediamine tetraacetic acid or metal salts thereof, 2-mercaptobenzimidazole, 3(2)-t-butyl-4-hydroxyanisole, 2,6-di-t-butyl-4-methylphenol, tetrakis[3-(3′,5′-di-t-butyl-4′-hydroxyphenyl)propionic acid]pentaerythritol, tocopherol acetate, rutin, quercetin, hydroquinone and metal thiosulfate salts.

5. The patch preparation according to claim 1, wherein said stabilizer comprises at least one or more species selected from the group consisting of ascorbic acid, metal salts or esters thereof, isoascorbic acid or metal salts, ethylenediamine tetraacetic acid or metal salts thereof, 2-mercaptobenzimidazole, 2,6-di-t-butyl-4-methylphenol, tetrakis[3-(3′,5′-di-t-butyl-4′-hydroxyphenyl)propionic acid]pentaerythritol, tocopherol acetate, rutin and metal thiosulfate salts.

6. The patch preparation according to claim 1, wherein said stabilizer comprises at least one or more species selected from the group consisting of ascorbic acid, metal salts or esters thereof, isoascorbic acid or metal salts thereof, 2-mercaptobenzimidazole, 2,6-di-t-butyl-4-methylphenol, metal salt of hydroxymethanesulfinic acid, rutin and metal metabisulfite salts.

7. The patch preparation according to claim 1, wherein said stabilizer comprises:
ascorbic acid, metal salts or esters thereof and metal metabisulfite salts, or
ascorbic acid, metal salts or esters thereof and 2-mercaptobenzimidazole.

8. The patch preparation according to claim 1, wherein said stabilizer comprises at least one or more species selected from the group consisting of ascorbic acid, metal salts or esters thereof, 2-mercaptobenzimidazole, metal metabisulfite salts and metal sulfite salts.

9. A patch preparation comprising, a support and a pressure-sensitive adhesive layer on at least one side of the support, the pressure-sensitive adhesive layer comprising donepezil, wherein said pressure-sensitive adhesive layer is obtainable by forming a film of a mixture comprising a pressure-sensitive adhesive, donepezil and a stabilizer,
and wherein said stabilizer comprises at least one or more species selected from the group consisting of ascorbic acid, metal salts or esters thereof, isoascorbic acid or metal salts thereof, ethylenediamine tetraacetic acid or metal salts thereof, cysteine, acetylcysteine, 2-mercaptobenzimidazole, 3(2)-t-butyl-4-hydroxyanisole, 2,6-di-t-butyl-4-methylphenol, tetrakis[3-(3′,5′-di-t-butyl-4′-hydroxyphenyl)propionic acid]pentaerythritol, 3-mercapto-1,2-propanediol, tocopherol acetate, rutin, quercetin, hydroquinone, metal salts of hydroxymethanesulfinic acid, metal metabisulfite salts, metal sulfite salts and metal thiosulfate salts.

10. The patch preparation according to claim 9, wherein said stabilizer comprises at least one or more species selected from the group consisting of ascorbic acid, metal salts or esters thereof, isoascorbic acid or metal salts thereof, ethylenediamine tetraacetic acid or metal salts thereof, cysteine, acetylcysteine, 2-mercaptobenzimidazole, 2,6-di-t-butyl-4-methylphenol, tetrakis[3-(3′,5′-di-t-butyl-4′-hydroxyphenyl)propionic acid]pentaerythritol, 3-mercapto-1,2-propanediol, tocopherol acetate, rutin, quercetin, metal salts of hydroxymethanesulfinic acid, metal metabisulfite salts, metal sulfite salts and metal thiosulfate salts.

11. The patch preparation according to claim 9, wherein said stabilizer comprises at least one or more species selected from the group consisting of ascorbic acid, metal salts or esters thereof, isoascorbic acid or metal salts thereof, ethylenediamine tetraacetic acid or metal salts thereof, 2-mercaptobenzimidazole, 2,6-di-t-butyl-4-methylphenol, tetrakis[3-(3′,5′-di-t-butyl-4′-hydroxyphenyl)propionic acid]pentaerythritol, tocopherol acetate, rutin, metal sulfite salts and metal thiosulfate salts.

12. The patch preparation according to claim 9, wherein said stabilizer comprises at least one or more species selected from the group consisting of ascorbic acid, metal salts or esters thereof, isoascorbic acid or metal salts thereof, ethylenediamine tetraacetic acid or metal salts thereof, 2-mercaptobenzimidazole, 3(2)-t-butyl-4-hydroxyanisole, 2,6-di-t-butyl-4-methylphenol, tetrakis[3-(3′,5′-di-t-butyl-4′-hydroxyphenyl)propionic acid]pentaerythritol, tocopherol acetate, rutin, quercetin, hydroquinone and metal thiosulfate salts.

13. The patch preparation according to claim 9, wherein said stabilizer comprises at least one or more species selected from the group consisting of ascorbic acid, metal salts or esters thereof, isoascorbic acid or metal salts, ethylenediamine tetraacetic acid or metal salts thereof, 2-mercaptobenzimidazole, 2,6-di-t-butyl-4-methylphenol, tetrakis[3-(3′,5′-di-t-butyl-4′-hydroxyphenyl)propionic acid]pentaerythritol, tocopherol acetate, rutin and metal thiosulfate salts.

14. The patch preparation according to claim 9, wherein said stabilizer comprises at least one or more species selected from the group consisting of ascorbic acid, metal salts or esters thereof, isoascorbic acid or metal salts thereof, 2-mercaptobenzimidazole, 2,6-di-t-butyl-4-methylphenol, metal salts of hydroxymethanesulfinic acid, rutin and metal metabisulfite salts.

15. The patch preparation according to claim 9, wherein said stabilizer comprises:
ascorbic acid, metal salts or esters thereof and metal metabisulfite salts, or
ascorbic acid, metal salts or esters thereof and 2-mercaptobenzimidazole.

16. The patch preparation according to claim 9, wherein said stabilizer comprises at least one or more species selected from the group consisting of ascorbic acid, metal salts or esters thereof, 2-mercaptobenzimidazole, metal metabisulfite salts and metal sulfite salts.
